(19) United States
(12) Patent Application Publication
(10) Pub. No.: US 2003/0211581 A1
(43) Pub. Date: Nov. 13, 2003

(54) Insulator element having enhancer-blocking properties

(76) Inventors: John C. Herr, Charlottesville, VA (US); P. Prabhakara Reddi, Charlottesville, VA (US)

Correspondence Address: John P Breen, University of Virginia Patent Foundation, Suite 1 110, 1224 West Main Street, Charlottesville, VA, 22903, US

(21) Appl. No.: 10/297,008

(22) PCT Filed: May 25, 2001
(86) PCT No.: PCT/US01/17110

(51) Int. Cl. C07H 21/04; C12P 21/04; C07K 14/47; C12N 5/06; C12N 15/85

(52) U.S. Cl. 435/69.7; 435/455; 435/320.1; 435/325; 530/350; 536/23.5

(57) ABSTRACT

The present invention relates to nucleic acid sequences that have insulator activity and the use of such sequences to prevent position effects on gene constructs transfected into cells. In one embodiment as shown in FIGS. 5A and 5B a vector is provided with a polylinker site (a) for insertion of a gene, wherein the polylinker site is flanked by one or more insulators (b). This insulator blocks enhancer effects on genes located downstream from the enhancer when the insulator is positioned between the enhancer and the gene.

This application claims priority under 35 U.S.C. §119(e) to provisional patent application No. 60/208,371, filed May 26, 2000, the disclosure of which is incorporated herein by reference in its entirety.

### US GOVERNMENT RIGHTS

This invention was made with United States Government support under Grant Nos. HD 36239 and HD 29099, awarded by the National Institutes of Health. The United States Government has certain rights in the invention.

### FIELD OF THE INVENTION

The present invention is directed to the isolation, identification, and characterization of cis-acting transcriptional control elements of higher eukaryotic gene promoters. More particularly, the invention is directed to the isolation of a DNA sequence that comprises a functional warm blooded vertebrate insulator element that blocks enhancer effects on the expression of adjacent genes.

### BACKGROUND OF THE INVENTION

The regulation of temporal and tissue-specific gene expression at the DNA level is mediated through an interaction between regulatory sequences in the DNA of eukaryotic cells and a complex of transcriptional factors (i.e. nucleoproteins) which are specific for a particular tissue type and for a particular gene. Further, the higher-order chromatin structure of tissue-specific genes is also regulated in a tissue-specific manner (reviewed by van Holde, K. E. (1989). “Chromatin structure and transcription”. In: Chromatin, K. E. van Holde, ed., New York, N.Y.; Springer-Verlag, pp. 355-408).

The cis-acting elements that control the temporal and spatial expression of a developmental or tissue-specific gene can be identified and linked to a other gene sequences (that are not normally associated with such regulatory elements) to regulate the expression of those genes. These techniques have been used to regulate the expression of therapeutic gene constructs. In particular, the regulatory elements of the developmental or tissue-specific gene can be isolated and linked to gene sequences, to express the gene product only in a particular tissue once the gene construct is introduced into a vertebrate species. Means for introducing novel gene constructs into vertebrate species are known to those skilled in the art. However, difficulties relating to controlled expression of transfected genes have been encountered.

One difficulty that has been encountered with the introduction of transgenic constructs into vertebrate species relates to clonal variation in the expression of the same gene in independent transformants. This problem is referred to as “position effect” variation and is thought to relate to the effects of DNA sequences adjacent to the site where the gene was inserted in the genome. In addition to variation of expression, sometimes the expression of the gene product is not regulated in accordance with the promoter used to express the gene. For example, expression of the gene product may occur in non-targeted tissues even though a tissue specific promoter was used to express the gene product. No completely satisfactory method of obviating these problems have yet been developed, and thus there is a continued need for a solution.

As noted above, problems relating to the controlled expression of introduced genes are believe to arise because the introduced gene has been inserted adjacent to regulatory elements normally present in the genome. For example it is known that enhancer elements can significantly increase the expression of adjacent genes. Thus if an introduced gene construct was inserted next to a strong enhancer element, the regulatory control of a tissue-specific promoter may be overridden by the enhancer, thus resulting in expression of the gene in non-targeted tissues.

Accordingly, to increase the predictability and safety of expressing foreign genes in vertebrate species a method must be provided that negates these “position effects.” The present invention is directed to nucleic acid sequences that block enhancer activity and other regulatory effects, thus allowing for a more predictable expression pattern for introduced gene constructs.

Insulators are nucleic acid sequences that function to block enhancer effects on genes, and therefore insulators can be used to block position effects and allow for better regulation of transfected genes. Insulator elements have been described in several nonvertebrate organisms, however, the isolation and functional characterization of such elements in higher vertebrates has been very limited. One vertebrate insulator sequence has been isolated from the chicken beta-globin gene, as described in U.S. Pat. No. 5,610,053. A similar insulator sequence is also present in humans, located about 20 kb upstream of the epsilon-globin gene and about 60 kb upstream of the beta-globin gene. However, this insulator element has shown limited utility as an insulator of enhancer activity. Accordingly, there is a need for additional vertebrate insulators that can shield foreign genes from position effects.

Definitions

In describing and claiming the invention, the following terminology will be used in accordance with the definitions set forth below.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH₂₋carbamate linkage (—CH₂OC(O)NR—), a phosphonate linkage, a —CH₂₋sulfonamide (—CH_{2—}S(O)₂NR—) linkage, a urea (—NHC(O)NH—) linkage, a —CH₂-secondary amine linkage, or with an alkylated peptidyl linkage (—C(O)NR—) wherein R is C₁₋C₄ alkyl;

2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁₋C₄ alkyl with the proviso that R and R₁ are not both hydrogen;

3. peptides wherein the C terminus is derivatized to —C(O)R₂ where R₂ is selected from the group consisting of C₁₋C₄ alkoxy, and —NR₃R₄ where R₃ and R₄ are independently selected from the group consisting of hydrogen and C₁₋C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Vat or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

As used herein, the term “conservative amino acid substitution” are defined herein as exchanges within one of the following five groups:

I. Small aliphatic, nonpolar or slightly polar residues:

Ala, Ser, Thr, Pro, Gly;

II. Polar, negatively charged residues and their amides:

Asp, Asn, Glu, Gln;

III. Polar, positively charged residues:

His, Arg, Lys;

IV. Large, aliphatic, nonpolar residues:

Met Leu, Ile, Val, Cys

V. Large, aromatic residues:

Phe, Tyr, Trp

As used herein, the term “purified” and like terms relate to the isolation of a molecule or compound in a form that is substantially free of contaminants normally associated with the molecule or compound in a native or natural environment.

The term “insulator” has its conventional meaning in the art, and refers to a DNA segment that insulates (i.e. moderates or negates the effects of regulatory elements adjacent to the gene) the transcription of genes placed within its range of action. In one context the insulator prevents enhancers located on one side of the insulator from acting on promoters located in the adjacent domain. Insulators are described in V. Corces, Nature 376, 462 (Aug. 10, 1995).

“Operably linked” refers to a juxtaposition wherein the components are configured so as to perform their usual function. For example, control sequences or promoters operably linked to a coding sequence are capable of effecting the expression of the coding sequence. More particularly, “an insulator that is operably linked to a gene” refers to an insulator that is covalently linked to a gene in the proper position and orientation to block the effect of an enhancer.

A “polylinker” is a nucleic acid sequence that comprises a series of three or more different restriction endonuclease recognitions sequences closely spaced to one another (i.e. less than 10 nucleotides between each site).

As used herein, the term “vector” is used in reference to nucleic acid molecules that transfer DNA segment(s) from one cell to another. Vectors are used to introduce foreign DNA into host cells where it can be replicated (i.e., reproduced) in large quantities. Examples of cloning vectors include plasmids, cosmids, lambda phage vectors, viral vectors (such as retroviral vectors).

As used herein a “gene” refers to the nucleic acid coding sequence as well as the regulatory elements necessary for the DNA sequence to be transcribed into messenger RNA (mRNA) and then translated into a sequence of amino acids characteristic of a specific polypeptide.

A promoter is a DNA sequence that directs the transcription of a DNA sequence, such as structural gene. Typically, a promoter is located in the 5′ region of a gene, proximal to the transcriptional start site of a structural gene. Promoters can be inducible (the rate of transcription changes in response to a specific agent), tissue specific (expressed only in some tissues), temporal specific (expressed only at certain times) or constitutive (expressed in all tissues and at a constant rate of transcription).

A core promoter contains essential nucleotide sequences for promoter function, including the TATA box and start of transcription. By this definition, a core promoter may or may not have detectable activity in the absence of specific sequences that enhance the activity or confer tissue specific activity. For example, the SP-10 core promoter consists of about 91 nucleotides 5′-ward of the transcriptional start site of the SP-10 gene, while the Cauliflower Mosaic Virus (CaMV) 35S core promoter consists of about 33 nucleotides 5′-ward of the transcriptional start site of the 35S genome.

An “enhancer” is a DNA regulatory element that can increase the efficiency of transcription, regardless of the distance or orientation of the enhancer relative to the start site of transcription.

As used herein a foreign gene refers to a DNA sequence that is operably linked to at least one heterologous regulatory element.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

“Therapeutic agent,” “pharmaceutical agent” or “drug” refers to any therapeutic or prophylactic agent which may be used in the treatment (including the prevention, diagnosis, alleviation, or cure) of a malady, affliction, disease or injury in a patient.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

### SUMMARY OF THE INVENTION

The present invention is directed to the isolation, identification, and characterization of cis-acting transcriptional control elements of higher eukaryotic gene promoters. More particularly, the present invention is directed to a 318-bp proximal portion of the promoter of the mouse SP-10 gene, and bioactive fragments thereof, that function as insulator elements to block enhancer effects on the expression of adjacent genes.

### BRIEF DESCRIPTION OF THE DRAWINGS

FIG. 1 represents an alignment of the mouse SP-10 promoter (SEQ ID NO: 9) with the corresponding human SP-10 promoter (SEQ ID NO: 10). Identical bases are indicated by a “|” symbol, whereas A to G (purine) and T to C (pyrimidine) substitutions indicated by a “:” symbol were considered similarities. The +1 indicates the transcription start site. Codons for the first eleven amino acids of the mouse and human SP-10 proteins are depicted as triplets, in italicized letters. Abbreviations: m=mouse; h=human.

FIG. 2 is a graph demonstrating that the CMV enhancer is sufficient to promote expression from the SP-10 core promoter (−91 to +28), and that the −408 to −92 fragment of the SP-10 promoter blocks CMV enhancer activity.

FIG. 3 is a graph demonstrating that the −408 to −92 fragment of the SP-10 promoter blocks CMV enhancer activity, but a stuffer fragment fails to block enhance activity.

FIG. 4 is a graph demonstrating that the enhancer-blocking activity of the SP-10 promoter maps within the −408 to −135 region. Abbreviations: CMV-91 luc=cytomegalovirus/core promoter/luciferase; CMV-408 luc=cytomegalovirus/−408 to +28 promoter/luciferase; CMV-266 luc=cytomegalovirus/−266 to +28 promoter/luciferase; CMV-186 luc=cytomegalovirus/−186 to +28 promoter/luciferase; CMV-135 luc=cytomegalovirus/−135 to +28 promoter/luciferase; and CMVst-91 luc=cytomegalovirus/stuffer fragment/core promoter/luciferase.

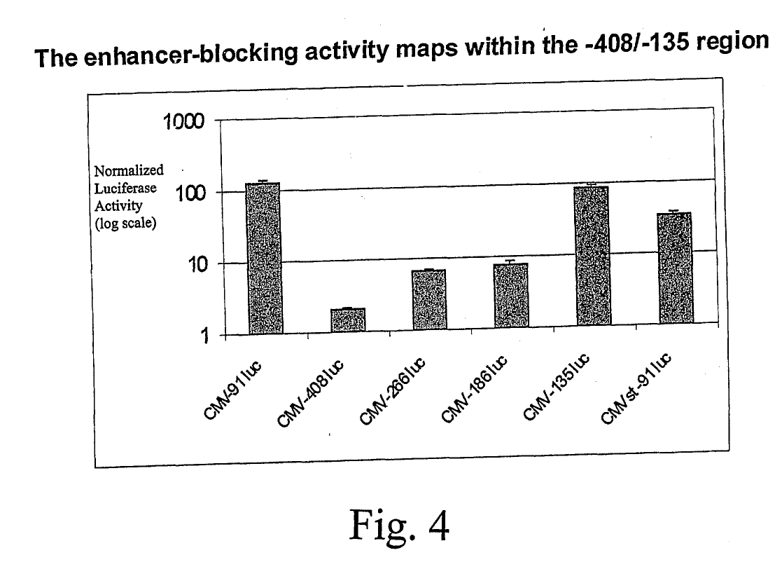

FIGS. 5A & 5B: FIG. 5A is a diagram of nucleic acid construct useful for minimizing position effects in transfection experiments, wherein a single insulator element is located on each end of the polylinker. FIG. 5B is a diagram of nucleic acid construct useful for minimizing position effects in transfection experiments, wherein a multiple insulator elements are located on each end of the polylinker. [symbol] represents an insulator element, [symbol] represents the polylinker sequence.

FIGS. 6A & 6B: FIG. 6A is a diagram of the nucleic acid construct used to produce transgenic mice. The construct comprises the CMV enhancer linked to the −408 to +28 region of the SP-10 promoter which in turn is operably linked to the Green Flourescent Protein (GFP). FIG. 6B represents RNA isolated from various tissues of the transgenic mouse: the upper panel represents an ethidium stained gel and the lower panel represents a Northern blot of that gel probed with a GFP probe. Lane 1 contains molecular weight markers and lanes 2-9 contain RNA isolated from brain, kidney, liver, lung, spleen, stomach, seminal vessicles and testis, respectively.

### DETAILED DESCRIPTION OF THE INVENTION

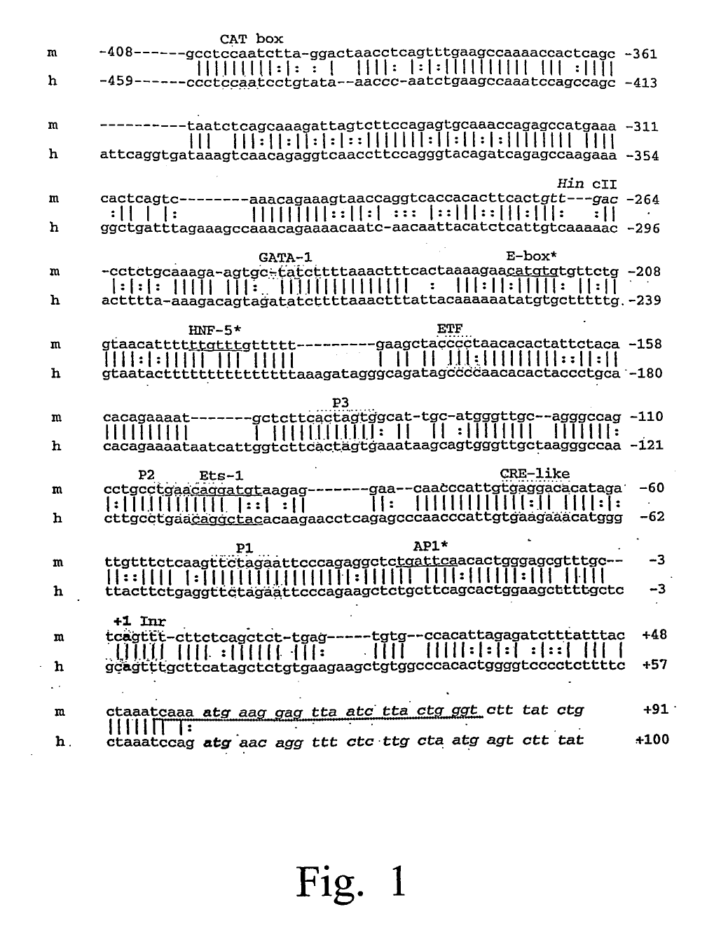

SP-10 is an acrosomal protein expressed in sperm of several species including human, mouse, monkey, baboon, fox, mouse and bull sperm. As shown in FIG. 1, a comparison of the mouse SP-10 promoter (SEQ ID NO: 9) with the corresponding homologous human promoter region (SEQ ID NO: 10) reveals a high degree of sequence identity between the human and mouse sequences. The −408 to +58 bp 5′ flanking region of mSP-10 shares 80% similarity with the −459 to +67 bp region of the human SP-10 gene. Identical bases are indicated by a “|” symbol, whereas A to G (purine) and T to C (pyrimidine) substitutions indicated by a “:” symbol were considered similarities. The +1 indicates the transcription start site. Conserved cis-acting elements which constituted recognition sequences for known transcription factors are indicated as well as three conserved palindrome sequences, designated P1, P2, and P3. An asterisk indicates those cis-acting elements present in the mouse gene alone. Codons for the first eleven amino acids of the mouse and human SP-10 proteins are depicted as triplets, in italicized letters.

The SP-10 gene is expressed only in sperm cells and only during a limited stage of development. In particular, mouse SP-10 gene transcription is restricted to step I-VII spermatids. Additional information regarding the SP-10 protein and its corresponding gene is provided in International Application Nos: PCT/US99/14275 and PCT/US01/01954, the disclosures of which are incorporated herein by reference.

Studies of the SP-10 promoter reveal that in transgenic mice the −408 to +28 bp region of SP-10 drives spermatid-specific transcription of a reporter gene, whereas the −91 to +28 bp core promoter lacks any promoter activity. Thus, the −408 to −91 bp region of the SP-10 promoter is critical for activation of gene expression in spermatids. Based on the observation that no transgenic lines have showed ectopic expression of a reporter gene in any somatic tissue tested, it was anticipated that the −408 to +28 bp SP-10 promoter may also play a role in down regulation of gene expression in somatic tissues. Thus the promoter region was analyzed for the presence of an insulator element that functions to prevent expression of the SP-10 gene product in somatic cells.

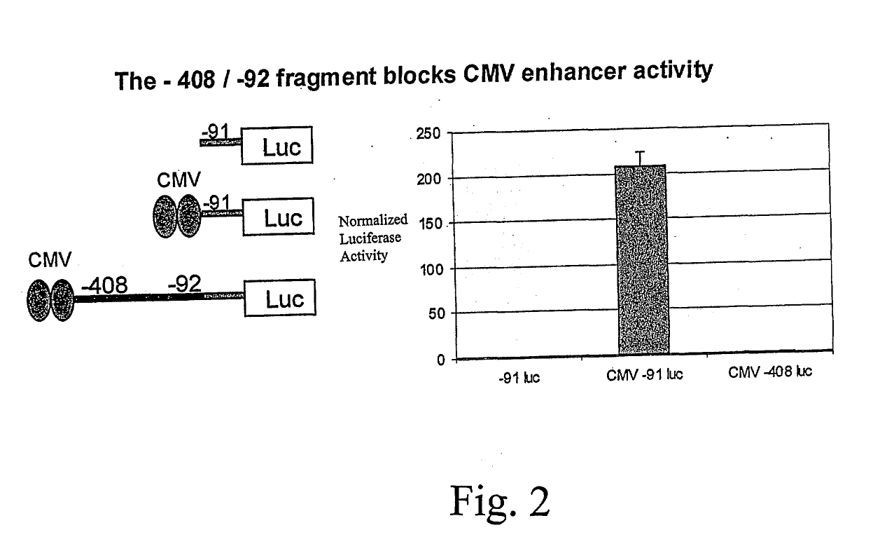

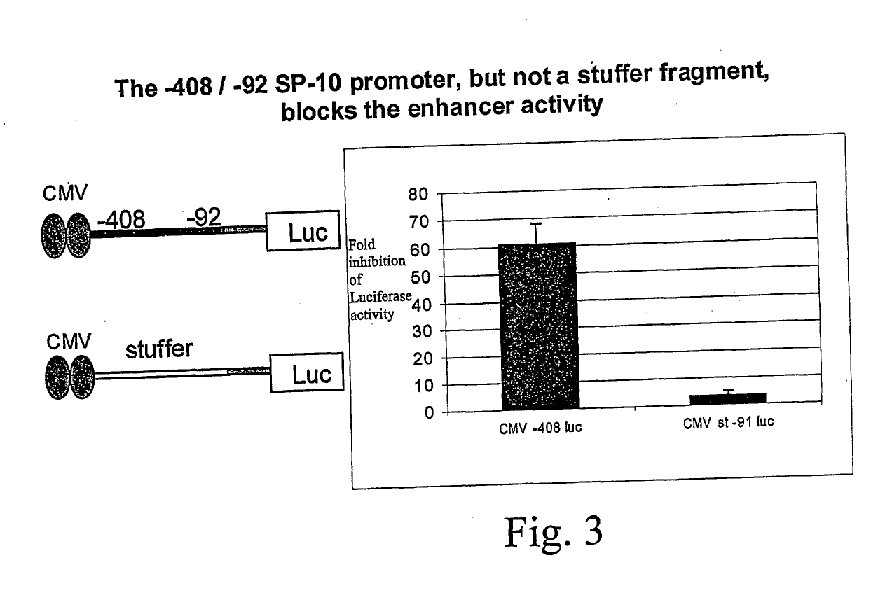

To determine if the −408 to +28 bp fragment of the SP-10 gene did contain an insulator element, the effect of the SP-10 promoter on transcription in somatic cells was examined in the context of a heterologous enhancer. When the cytomegalovirus (CMV) enhancer was placed upstream of the SP-10 core promoter (−91 to +28 bp); a robust luciferase activity was observed in COS-7 cells, suggesting that the SP10 core promoter element in combination with an enhancer can initiate transcription in somatic cells. However, when the −408 to −91 bp fragment of the SP-10 promoter was inserted between the CMV enhancer and the SP-10 core promoter an approximately 78-fold reduction in luciferase activity was observed (See FIG. 2). Two separate control stuffer fragments caused only a 4-fold reduction (See FIG. 3) suggesting that the −408 to +28 bp SP-10 promoter functions as a strong repressor of transcription in somatic cells.

Repressor activity was observed only when the −408 to −91 bp fragment was placed between the CMV enhancer and the core promoter but not upstream of the enhancer. These data clearly demonstrate that the −408 to +28 bp SP-10 proximal promoter induces transcriptional repression in somatic cells, thereby suggesting a possible mechanism for the position-independent repression of the reporter gene in somatic tissues of transgenic mice. Accordingly, the present invention is directed to an isolated or purified nucleic acid sequence comprising an insulator sequence, wherein said insulator sequence consists essentially of the −408 to −92 region of the mouse SP-10 promoter (SEQ ID NO: 1) or the corresponding region of the human homolog (SEQ ID NO: 3), or an insulator active fragment derived from those sequences such as SEQ ID NO: 2, SEQ ID NO: 4, SEQ ID NO: 7 or SEQ ID NO: 8.

Analysis of the SP-10 promoter fragments that retain insulator activity (i.e. the −186 to −92 fragment) with an SP-10 fragment that lacks activity (i.e. the −135 to −92 fragment) reveals a repeated motif (ACACAC; SEQ ID NO: 11) that is conserved between the mouse and human sequences. Therefore it is anticipated that this sequence plays an important role in the function of the insulator region. In accordance with one embodiment of the present invention an insulator element is provided that comprises one or more ACACAC (SEQ ID NO: 11) sequences. In one embodiment the insulator comprises the sequence ACACACNNNNNNACACAC (SEQ ID NO: 12), wherein N represents any nucleotide selected from the group consisting of adenosine, threonine, cytosine and guanidine.

In one embodiment an isolated insulator DNA molecule is provided, wherein the insulator consists of a eukaryotic sequence isolated from the human or mouse SP-10 gene, more particularly from SEQ ID NO: 9 or SEQ ID NO: 10. This sequence is a chromatin insulator which when flanking a gene to be inserted into a host chromosome insulates the transcriptional expression of said gene from one or more cis-acting regulatory sequences present in the chromatin into which the gene has been inserted. In one embodiment the insulator has the nucleotide sequence of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

One aspect of the present invention pertains to compositions and methods for insulating the expression of a gene in higher eukaryotic organisms, including humans, through the use of the isolated insulator element of the present invention. Accordingly, the present invention encompasses various nucleic acid constructs and vectors that comprise the insulator element of the present invention and the use of such constructs to produce transgenic cells and organisms.

In one embodiment a nucleic acid construct is provided wherein the insulator element is operably linked to a polylinker. The polylinker comprises a series of endonuclease restriction sites in close proximity to one another and provides a convenient method for inserting a gene into the construct adjacent to the insulator. Polylinkers are well known to those skilled in the art and typically comprise three or more different restriction sites that are unique to the vector construct. In this manner a gene sequence that is operably linked to a tissue specific promoter can be inserted into the nucleic acid construct. The resulting construct can then be used to transfect cells, and the insulator element of the construct will protect the expression of that gene from position effects.

In one preferred embodiment the nucleic acid construct is a viral vector or plasmid and the insulator element has the sequence of SEQ ID NO: 1, SEQ ID NO: 2, SEQ ID NO: 3 or SEQ ID NO: 4. In a further embodiment, the viral vector or plasmid construct comprises multiple copies of the insulator in the form of tandem repeats arranged in a head to tail orientation. The tandem repeats may optionally include spacer nucleic acid sequences (i.e. nucleotides that do not function as part of the insulator) ranging from 3 to 50 nucleotides, more preferably from about 3 to about 20, and in one embodiment the spacer is 6 nucleotides in length. The vector constructs can also be provided with a selectable marker or reporter gene that allows for the identification of cells containing the vector.

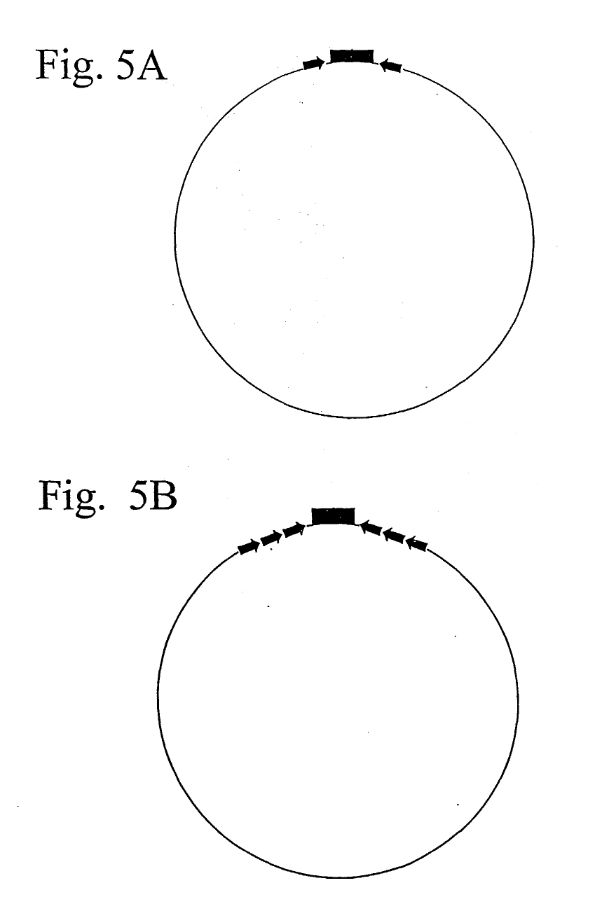

In an alternative embodiment, the construct comprises a polylinker that is flanked at either end by insulator elements of the present invention. In one embodiment the vector, preferably a plasmid, comprises a first insulator operably linked to the first end of a polylinker and a second insulator element operably linked to the second end of the polylinker, such that the polylinker is flanked on each end by an insulator element (see FIG. 5A). In this construct the first and second insulators are sequences independently selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In a further embodiment, the first and second insulators, comprise multiple copies of a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8 linked together in tandem and in a head to tail orientation (see FIG. 5B). The tandem repeats may optionally include spacer nucleic acid sequences (i.e. nucleotides that do not function as part of the insulator) ranging from 3 to 50 nucleotides, more preferably from about 3 to about 20, and in one embodiment the spacer is 6 nucleotides in length.

Preferred insulator elements of this invention consist of a nucleic acid sequence selected from the group consisting of SEQ ID NO: 1, SEQ ID NO: 2, SEQ ID NO: 3, SEQ ID NO: 4, SEQ ID NO: 7, and SEQ ID NO: 8, or fragments of those sequences having insulator activity. The present invention also encompasses sequences substantially homologous to SEQ ID NO: 1, SEQ ID NO: 2, SEQ ID NO: 3, SEQ ID NO: 4, SEQ ID NO: 7, and SEQ ID NO: 8 such that they will hybridize to those sequences under stringent conditions and have insulator activity. In one embodiment the claimed insulator sequence comprises a nucleic acid sequence of 300 nucleotides or less that hybridizes to a nucleotide fragment of SEQ ID NO: 1 or SEQ ID NO: 3, or their respective complements under stringent conditions.

The hybridizing portion of the hybridizing nucleic acids is typically at least 15 (e.g., 20, 25, 30, 50 or 100) nucleotides in length. Hybridizing nucleic acids of the type described herein can be used, for example, as a cloning probe, a primer (e.g., a PCR primer), or a diagnostic probe. Accordingly, the nucleic acid sequences of SEQ ID NO: 1, SEQ ID NO: 3 or fragments of those sequences can be used as probes to detect additional genes that are regulated by similar insulator elements. It is anticipated that DNA sequences that hybridize to SEQ ID NO: 1, SEQ ID NO: 3 or fragments thereof under stringent or highly stringent conditions will also have insulator activity.

As used herein, the term “probe” refers to an oligonucleotide (i.e., a sequence of nucleotides), that is capable of hybridizing to another oligonucleotide of interest. The probe may be single-stranded or double-stranded. It is contemplated that any probe used in the present invention will be labeled with any “reporter molecule,” which provides a detectable signal in any detection system, including, but not limited to fluorescent, enzyme (e.g., ELISA, as well as enzyme-based histochemical assays), radioactive, and luminescent systems.

Nucleic acid duplex or hybrid stability is expressed as the melting temperature or Tm, which is the temperature at which a nucleic acid duplex dissociates into its component single stranded DNAs. The equation for calculating the Tm of nucleic acids is well known in the art. As indicated by standard references, a simple estimate of the Tm value may be calculated by the equation: Tm (° C.)=81.5+0.41(% G+C), when a nucleic acid is in aqueous solution at 1M NaCl (see e.g., Anderson and Young, Quantitative Filter Hybridization, in Nucleic Acid Hybridization (1985)). Other references include more sophisticated computations which take into account the length of the probe, as well as structural and sequence characteristics into account for the calculation of Tm.

This melting temperature is used to define the stringency conditions of the hybridization and washes for hybridization reactions. Typically a 1% mismatch results in a 1° C. decrease in the Tm, and the temperature of the final wash in the hybridization reaction is reduced accordingly (for example, if two sequences have >95% identity, the final wash temperature is decreased from the Tm by 5° C.). In practice, the change in Tm can be between 0.5° C. and 1.5° C. per 1% mismatch.

In accordance with one embodiment, the present invention is directed to the nucleic acid sequence of SEQ ID NO: 1 or SEQ ID NO: 3 and nucleic acid sequences that hybridize to those sequences (or fragments thereof) under stringent or highly stringent conditions. In accordance with the present invention highly stringent conditions are defined as conducting the hybridization and wash conditions at no lower than −5° C. Tm. Stringent conditions are defined as involve hybridizing at 68° C. in 5×SSC/5×Denhardt's solution/1.0% SDS, and washing in 0.2×SSC/0.1% SDS at 68° C. Moderately stringent conditions include hybridizing at 68° C. in 5×SSC/5×Denhardt's solution/1.0% SDS and washing in 3×SSC/0.1% SDS at 42° C. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

In accordance with one embodiment the insulator of the present invention is used to insulate or buffer the expression of a gene from the action of an adjacent regulatory element, such as an enhancer, after a foreign gene is inserted into a cell's DNA. The method comprises the step of operably linking a insulator element upstream of the promoter of said gene prior to introducing the gene into the cell. In one embodiment the gene of interest is flanked at both ends by one or more insulator sequences. In one embodiment the gene of interest is inserted into the polylinker site of a transfection vector, wherein the polylinker of the vector is flanked by one or more insulator elements. In addition, the vector can be further provided with. a selectable marker gene. Once the gene has been inserted into the polylinker of the vector and operably linked to the insulator element(s), the construct is transfected into a cell in vitro or in vivo using techniques known to those skilled in the art. Accordingly, in one embodiment of the present invention the method for insulating the expression of an introduced gene from cis-acting DNA sequence regulatory elements present in the chromatin into which the gene has integrated comprises the following steps. Transfecting a vector into a cell, wherein the vector comprises an insulator of the present invention operably linked to a gene, and integrating the construct into the chromatin of the cell. Expression of the resultant integrated heterologous gene is then insulated from any cis-acting DNA regulatory sequences present in the chromatin of the cell.

The insulator element-containing constructs of the present invention allow for a more precise control of expression of foreign genes after the transfection of cells due to the reduced impact/elimination of position effects. In particular, the present invention provides a nucleic acid construct and method for insulating the expression of a gene or genes in transgenic animals such that the transfected genes will be protected and stably expressed in the tissues of the transgenic animal or its offspring. For example, the transfected genes will be expressed even if the DNA of the construct integrates into areas of silent or active chromatin in the genomic DNA of the host animal.

In accordance with one embodiment, a vector is provided that comprises an insulator, a tissue specific promoter and a polylinker, wherein the insulator is operably linked to the promoter and the promoter is operably linked to the polylinker so that when the coding sequence of a gene is inserted at the polylinker site the gene is transcribed under the control of the promoter. Optionally, the insulator elements, reporter gene(s), and polylinker region may be provided in the form of a cassette designed to be conveniently ligated into a suitable plasmid or vector, which plasmid or vector is then used to transfect cells or tissues, and the like, for both in vitro and in vivo use.

The present insulator element promises to be a useful tool in regulating the expression of foreign genes introduced into cells in vivo or in vitro, including applications relating to gene therapy and gene transfer techniques. Use of the present insulator as a boundary element ensures strict tissue-specific expression of the required gene. For example, an insulator can be operably linked to a therapeutic gene to prevent the negative effects on the transduced gene caused by the site of integration. Thus corrected versions of genes can be introduced into stem cells in vitro or in vivo to provide alterations to those cells. The incorporation of insulator sequences in constructs used for cell transfection would ensure expression of transgenes in a predictable manner—an important aspect for research purposes as well gene therapy applications.

In accordance with one embodiment a kit is provided for transfecting cells. To this end, vectors and cells comprising the insulator element of the present invention operatively linked to a polylinker or gene can be packaged in a variety of containers, e.g., vials, tubes, microtiter well plates, bottles, and the like. In one embodiment a kit containing the vector constructs of the invention is provided for use to insulate the expression of a transfected gene or genes integrated into host DNA. Other reagents can be included in separate containers and provided with the kit; e.g., positive control samples, negative control samples, buffers, cell culture media, etc.

In one embodiment of the present invention, therapeutic genes can be inserted into expression vectors that contain the insulator elements of the present invention and used to transfect cells to ensure the correct expression of the gene in the target cells. In accordance with one embodiment, the gene is operably linked to one or more insulator elements and the construct is transfected into and expressed in a eukaryotic host cell. Suitable eukaryotic host cells and vectors are known to those skilled in the art. In particular, nucleic acid sequences may be introduced into a cell or cells in vitro or in vivo using delivery mechanisms such as liposomes, viral based vectors, or microinjection.

The present invention is also directed to the transgenic host cells and non-human transgenic organisms produced using DNA constructs comprising a nucleic acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In one embodiment the transgenic host cell comprises a nucleic acid construct that includes an insulator sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8 operably linked to a recombinant gene. Preferably, the construct is inserted into the genome of the host cell, and the recombinant gene comprises a tissue specific promoter linked to a non-native coding sequence (i.e. the tissue specific promoter is not naturally associated with the coding sequence). In one embodiment the vector used to transform the cells comprises one or more isolated insulator DNA molecules selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8 operably linked to a promoter domain, which in turn is operably linked to a heterologous gene and the gene is optionally linked to an insulator positioned 3′ of the gene.

Host cells are selected from eukaryotic cells including plant and animal cells. Preferably the host cell is a vertebrate species host cell, and more preferably the cell is from a warm-blooded vertebrate species. In one embodiment, the host cells are selected from human, primate or mouse cells. Methods for transforming host cells are well known to those skilled in the art and vary with the type of cell to be transformed. In one embodiment of the present invention, a recombinant host cell is provided wherein the cell contains a heterologous DNA construct comprising an expression cassette, wherein the cassette comprises, in the 5′ to 3′ direction, an insulator sequence of the present invention, a transcription initiation region operably linked to the coding region of a structural gene and an optional second insulator sequence.

### EXAMPLE 1

### Identification of the SP-10 Insulator

Insulators are DNA sequences with enhancer-blocking properties. The observation that a 436-bp proximal promoter fragment (−408 to +28) of the SP-10 gene directed position-independent cell-type specificity of transcription in several transgenic lines led to the hypothesis that, in addition to positive regulatory elements responsible for spermatid expression, the 436-bp promoter must also contain sequences that actively prevented activation of the transgene in somatic cells.

To test this hypothesis, luciferase constructs were prepared (pGL3 vector, Promega, WI) in which a strong CMV enhancer (bp-59 to 465 in pEGFPN1, Clontech, CA) was placed in front of 1) core promoter of SP-10 gene (−91 to +28 bp), and 2) the 436-bp SP-10 proximal promoter (−408 to +28). In transiently transfected COS-7 cells, circular plasmid with a CMV enhancer placed upstream of the SP-10 core promoter produced a robust luciferase activity. However, this activity was inhibited 70-fold when the CMV enhancer was placed upstream of the −408 to +28 bp promoter (See FIG. 2). In a control plasmid in which a stuffer fragment replaced the −408 to −91 region, only a 3-fold inhibition was observed (FIG. 3). Two different DNA sequences were used as the stuffer fragment. In one experiment the stuffer fragment comprised a portion of the β-lactamase gene and in a second experiment the stuffer fragment was a portion of the cloning vector pCR®-TOPO (Invitrogen Corporation). This experiment demonstrates that sequences upstream of the SP-10 core promoter (−408 to −91) possess an enhancer-blocking activity in COS-7 cells.

To test the generality of this enhancer-blocking property, the experiment was repeated using a number of different somatic cell lines. The −408 to −91 SP-10 promoter fragment blocked enhancer activity in all the cell-lines tested, including CHO (ovary), NIH3t3 (fibroblast), HEK (kidney), TM3 (Leydig)) or TM4 (Sertoli) cells.

Two conclusions emerge from these experiments. 1. The SP-10 core promoter (−91 to +28 bp) can initiate transcription in somatic cells in the context of a heterologous enhancer. 2. More importantly, the SP-10 proximal promoter (−408 to −91) possesses an enhancer blocking activity.

### EXAMPLE 2

### Identification of the Minimal SP-10 Insulator Sequence

In order to map the enhancer-blocking activity to a smaller region, a systematic set of deletions from the 5′ end of the proximal promoter were performed (See FIG. 3). The −266 to +28 bp, and the −186 to +28 bp fragment retained enhancer-blocking ability (approximately a 20-fold and 17-fold reduction in transcription was observed, respectively), albeit to a lesser degree compared to the −408 to +28 bp fragment. In contrast, the −135 to +28 bp promoter fragment showed only a 3-fold inhibition of enhancer action, suggesting that the SP-10 promoter region between −408 to −135 bp contains the sequences responsible for enhancer-blocking activity.

The insulator functions in a directional and orientation specific manner. In particular the insulator must be positioned between the enhancer and the gene to block enhancer effects on the gene. In addition the insulator functions best when it is inserted in the orientation as found in the native SP-10 gene. Inverting the sequence results in only a 10 fold reduction in transcription relative to a 70 fold reduction in the native orientation.

### EXAMPLE 3

### Insulation of Expression in a Transgenic Mouse

Generation of Transgenic Mice

Transgenic mice were produced by microinjection of transgenes into the male pronuclei of fertilized mouse eggs and transfer of these eggs to psuedopregnant foster mothers using standard procedures (Hogan et al.,1986, Manipulating the Mouse Embryo. Cold Spring Harbor Press, NY). Transgenic mice were generated by the Transgenic Mouse Core Facility (TMCF) at the University of Virginia. Transgenic founder mice were identified by two methods. (1) Tail DNA was subjected to PCR amplification using GFP primers; 5′ GTGAGCAAGGGCGAGGAGCTG (SEQ ID NO:13) (nt # 100-120, GenBank accession #U 55761) and 5′ CTTGTACAGCTCGTCCATGCCGAG (SEQ ID NO:14) (nt# 8 13-790, Genbank accession #U55761) spanning a 713 bp portion of the GFP cDNA. The amplification of 713 bp gfp PCR product identified the founder mice. (2) Transgene integration was confirmed by Southern hybridization (Sambrook et al., 1989, Molecular Cloning: A Laboratory Manual, 2nd ed., Cold Spring Harbor Laboratory Press, NY.). Mouse genomic DNAs were cut with EcoR I, transferred to nylon membrane (Stratagene, La Jolla, Calif.), and probed with radiolabeled GFP cDNA (derived from pEGFP 1). The founder mice were identified on the basis of positive hybridization band(s) obtained with the gfp probe. The positive founders were mated with C57 B1 partners to obtain an F1 generation for analysis.

Testing of the Insulator Function in Transgenic Mice

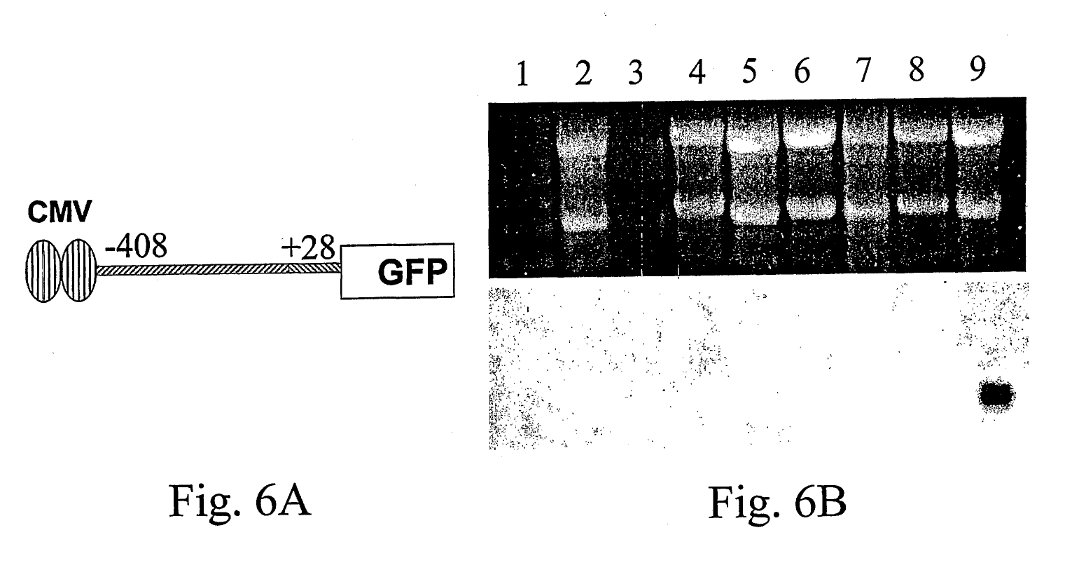

The insulator function of the SP-10 promoter was tested in the context of chromatin by generating transgenic mice. The mice were transfected with a construct comprising a CMV enhancer placed upstream of the −408/+28 mouse SP-10 promoter, with the promoter operably linked to a GFP reporter (see FIG. 6A). This construct was used to mimic the intergration of the transgene adjacent to a potent transcriptional enhancer. Two lines of transgenic mice (6623 and 7093) that showed evidence of integration of the CMV+SP-10 promoter+GFP DNA were analyzed for GFP expression. Northern analysis indicated the presence of GFP mRNA in testis but not in several somatic tissues including brain, heart, liver, lung, kidney, muscle, intestine, seminal vesicle and prostate (see FIG. 6B). This result indicated that the CMV enhancer, which is otherwise capable of driving transcription in somatic tissues, was prevented by the SP-10 promoter from activating transcription.

In addition, both transgenic lines showed evidence of GFP expression in the round spermatids. Seminiferous tubules from the testis of a 35 day old transgenic mouse (#6623 line) were examined by the transillumination assisted microdissection method. At day 35, mouse testis consists of meiotic as well as postmeiotic cells. Bright fluorescent round spermatids indicated the expression of GFP, whereas spermatocytes did not express GFP.

Taken together, the data showed that the −408/+28 SP-10 promoter functions as enhancer blocker in a direction and position dependent way when tested episomally in different cell lines. The enhancer-blocking insulator activity was also demonstrated in the context of chromatin through the use of transgenic mice that contained a CMV+SP-10 promoter+GFP DNA construct inserted into the genome.

1. An isolated or purified nucleic acid sequence comprising an insulator sequence wherein said insulator sequence consists essentially of a nucleic acid sequence selected from the group consisting of SEQ ID NO: 1, SEQ ID NO: 2, SEQ ID NO: 3, SEQ ID NO: 4, SEQ ID NO: 7, and SEQ ID NO: 8.

2. A nucleic acid construct comprising an insulator element operably linked to a polylinker, wherein said insulator element consists of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

3. The nucleic acid construct of claim 2 wherein said insulator element consists of SEQ ID NO: 1, SEQ ID NO: 2, SEQ ID NO: 3, or SEQ ID NO: 4.

4. The nucleic acid construct of claim 2 wherein said insulator element consists of SEQ ID NO: 7 or SEQ ID NO: 8.

5. The nucleic acid construct of claim 2 wherein the construct further comprises a second insulator element operably linked to the polylinker such that the polylinker is flanked on each end by an insulator element, said second insulator element consists of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

6. The nucleic acid construct of claim 5 wherein said second insulator element consists of SEQ ID NO: 1, SEQ ID NO: 2, SEQ ID NO: 3, or SEQ ID NO: 4.

7. The nucleic acid construct of claim 2 wherein said construct is a plasmid.

8. The nucleic acid construct of claim 5 wherein said construct is a plasmid.

9. A nucleic acid construct comprising two or more insulator elements operably linked to a polylinker, wherein said insulator elements are linked to one another in tandem repeats and each insulator element consists of a nucleic acid sequence independently selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

10. The nucleic acid construct of claim 9 wherein the polylinker is flanked by a second set of two or more insulator elements, wherein said second set of insulator elements are linked together in tandem repeats and operably linked to said polylinker, each of said second insulator element consisting of a nucleic acid sequence independently selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

11. A nucleic acid sequence of 400 nucleotides or less that hybridizes to a 50 nucleotide fragment of SEQ ID NO: 1 or SEQ ID NO: 2 under stringent conditions.

12. A transgenic host cell comprising the nucleotide sequence of claim 11.

13. A method of decreasing the influence of adjacent sequences on the expression of a gene after insertion of said gene into a cell's DNA, said method comprising the step of operably linking an insulator element upstream of the promoter of said gene prior to introducing the gene into the cell.

14. The method of claim 13 further comprising the step of operably linking an insulator element downstream from the coding region of said gene.

15. A transgenic host cell comprising a nucleic acid construct, wherein said construct comprises an insulator sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8 operably linked to a recombinant gene.

16. The transgenic host cell of claim 15 wherein said recombinant gene comprises a tissue specific promoter linked to a non-native coding sequence.


SEQUENCE LISTING (14 sequences)

SEQ ID NO: 1 - DNA, 318 bp, Mus musculus
gcctccaatcttaggactaacctcagtttgaagccaaaaccactcagctaatctcagcaaagattagtcttccagagtgcaaaccagagccatgaaacactcagtcaaacagaaagtaaccaggtcaccacacttcactgttgaccctctgcaaagaagtgctatcttttaaactttcactaaaagaacatgtgtgattctggtaacattttttgtttgtttttgaagctacccctaacacactattctacacacagaaaatgctcttcactagtggcattgcatgggttgcagggccagcctgcctgaacaggatgt

SEQ ID NO: 2 - DNA, 357 bp, Homo sapiens
ccctccaatcctgtataaacccaatctgaagccaaatccagccagcattcaggtgataaagtcaacagaggtcaaddttccagggtacagatcagagccaagaaaggctgatttagaaagccaaacagaaaacaatcaacaattacatctcattgtcaaaaacacttttaaaagacagtagatatcttttaaactttattacaaaaaatatgtgctttttggtaatactttttttttttttttaaagatagggcagatagccccaacacactaccctgcacacagaaaataatcattggtcttcactagtgaaataagcagtgggttgctaagggccaacttgcctgaacaggctac

SEQ ID NO: 3 - DNA, 105 bp, Homo sapiens
gcagatagccccaacacactaccctgcacacagaaaataatcattggtcttcactagtgaaataagcagtgggttgctaagggccaacttgcctgaacaggctac

SEQ ID NO: 4 - DNA, 94 bp, Mus musculus
gaagctacccctaacacactattctacacacagaaaatgctcttcactagtggcattgcatgggttgcagggccagcctgcctgaacaggatgt

SEQ ID NO: 5 - DNA, 275 bp, Mus musculus
gcctccaatcttaggactaacctcagtttgaagccaaaaccactcagctaatctcagcaaagattagtcttccagagtgcaaaccagagccatgaaacactcagtcaaacagaaagtaaccaggtcaccacacttcactgttgaccctctgcaaagaagtgctatcttttaaactttcactaaaagaacatgtgtgattctggtaacattttttgtttgtttttgaagctacccctaacacactattctacacacagaaaatgctcttcactagt

SEQ ID NO: 6 - DNA, 310 bp, Homo sapiens
ccctccaatcctgtataaacccaatctgaagccaaatccagccagcattcaggtgataaagtcaacagaggtcaaddttccagggtacagatcagagccaagaaaggctgatttagaaagccaaacagaaaacaatcaacaattacatctcattgtcaaaaacacttttaaaagacagtagatatcttttaaactttattacaaaaaatatgtgctttttggtaatactttttttttttttttaaagatagggcagatagccccaacacactaccctgcacacagaaaataatcattggtcttcactagt

SEQ ID NO: 7 - DNA, 51 bp, Mus musculus
gaagctacccctaacacactattctacacacagaaaatgctcttcactagt

SEQ ID NO: 8 - DNA, 58 bp, Homo sapiens
gcagatagccccaacacactaccctgcacacagaaaataatcattggtcttcactagt

SEQ ID NO: 9 - DNA, 500 bp, Mus musculus
gcctccaatcttaggactaacctcagtttgaagccaaaaccactcagctaatctcagcaaagattagtcttccagagtgcaaaccagagccatgaaacactcagtcaaacagaaagtaaccaggtcaccacacttcactgttgaccctctgcaaagaagtgctatcttttaaactttcactaaaagaacatgtgtgattctggtaacattttttgtttgtttttgaagctacccctaacacactattctacacacagaaaatgctcttcactagtggcattgcatgggttgcagggccagcctgcctgaacaggatgtaagaggaacaacccattgtgaggacacatagattgtttctcaagttctagaattcccagaggctctgattcaacactgggagcgtttgctcagtttcttctcagctcttgagtgtgccacattagagatctttatttacctaaatcaaaatgaaggagttaatcttaatgggtctttatctg

SEQ ID NO: 10 - DNA, 559 bp, Homo sapiens
ccctccaatcctgtataaacccaatctgaagccaaatccagccagcattcaggtgataaagtcaacagaggtcaaccttccagggtacagatcagagccaagaaaggctgatttagaaagccaaacagaaaacaatcaacaattacatctcattgtcaaaaacacttttaaaagacagtagatatcttttaaactttattacaaaaaatatgtgctttttggtaatactttttttttttttttaaagatagggcagatagccccaacacactaccctgcacacagaaaataatcattggtcttcactagtgaaataagcagtgggttgctaagggccaacttgcctgaacaggctacacaagaacctcagagcccaacccattgtgaagaaacatgggttacttctgaggttctagaattcccagaagctctgcttcagcactggaagcttttgctcgcagtttgcttcatagctctgtgaagaagctgtggcccacactggggtcccctcttttcctaaatccagatgaacaggtttctcttgctaatgagtctttat

SEQ ID NO: 11 - DNA, 6 bp, Mus musculus
acacac

SEQ ID NO: 12 - DNA, 18 bp, Mus musculus
  misc_feature (7)..(7): “n” represent any amino acid
acacacnnnnnnacacac

SEQ ID NO: 13 - DNA, 21 bp, Artificial Sequence
  primer for green fluorescent protein
gtgagcaagggcgaggagctg

SEQ ID NO: 14 - DNA, 24 bp, Artificial Sequence
  primer for green fluorescent protein
cttgtacagctcgtccatgccgag